(19) United States
(12) Patent Application Publication
(10) Pub. No.: US 2016/0312234 A1
(43) Pub. Date: Oct. 27, 2016

(54) DETERMINING USER CHARGES USING TRACKING INFORMATION

(71) Applicant: Hal R. Varian, Danville, CA (US)

(72) Inventor: Hal R. Varian, Danville, CA (US)

(73) Assignee: GOOGLE INC., Mountain View, CA (US)

(21) Appl. No.: 11/770,339

(22) Filed: Jun. 28, 2007

(51) Int. Cl. G06Q 30/02; G06Q 30/00

(52) U.S. Cl. CPC C12N 15/82

(57) ABSTRACT

The present disclosure includes a system and method for determining user charges using tracking information. In some implementations, a method for charging advertisers includes identifying tracking information associated with ads positioned in Web pages in accordance with a position auction. The ads positioned in the Web pages are based, at least in part, on associated bid amounts. The charges for agents associated with the ads are determined using the tracking information and the bid amounts. The charges are determined independent of position normalizers. Invoices for the agents are generated including the charges for presenting the ads.

### TECHNICAL FIELD

This invention relates to advertisements.

### BACKGROUND

Content delivery over the Internet continues to improve every day. Computer users can receive e-mail, news, games, entertainment, music, books, and web pages—all with a simple Internet connection (and with improved quality on a broadband connection). Internet users also have access to a plethora of services such as maps, shopping links, images, blogs, local search, satellite images, group discussions, hosted content, and e-mail. While many of these services are free to users, such services are often accompanied by an advertisement (“ad”) that helps service providers defray the cost of providing these services. In addition, the advertisement may also add value to the user experience.

### SUMMARY

The present disclosure includes a system and method for determining user charges using tracking information. In some implementations, a method for charging advertisers includes identifying tracking information associated with ads positioned in Web pages in accordance with a position auction. The ads positioned in the Web pages are based, at least in part, on associated bid amounts. The charges for agents associated with the ads are determined using the tracking information and the bid amounts. The charges are determined independent of position normalizers. Invoices for the agents are generated including the charges for presenting the ads.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a block diagram illustrating an example payment system for position auction bidders;

FIGS. 2A and 2B are matrices for determining payments in the system of FIG. 1;

FIGS. 3A and 3B are other matrices for determining payments in the system of FIG. 1;

FIG. 4 is a flow diagram illustrating an example method for determining charges in a position auction; and

FIG. 5 is a flow diagram illustrating another example method for determining charges in a position auction.

Like reference symbols in the various drawings indicate like elements.

### DETAILED DESCRIPTION

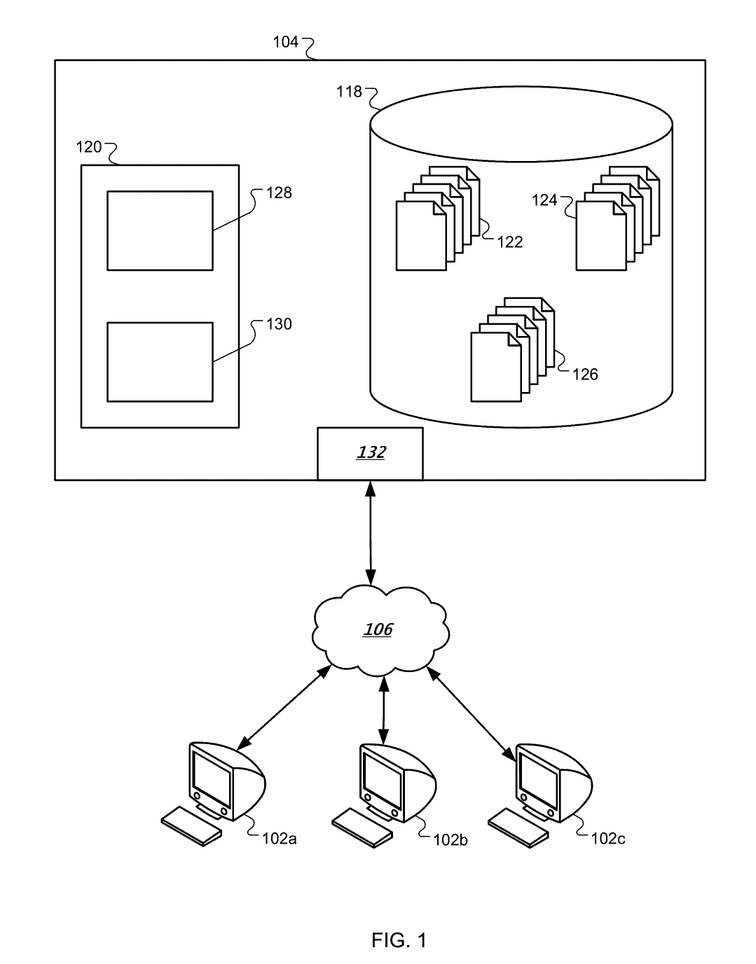

FIG. 1 illustrates a billing system 100 for determining charges in a position auction independent of position normalizers. A position normalizer can include an estimate of a metric associated with an advertisement (e.g., estimated click-through rate) used to determine expected costs for advertising. For example, estimated click through rates can be used to determine the expected cost for presenting an ad in a Web page. In a conventional position auction, agents (e.g., advertisers or their proxies) bid on positions in Web pages for presenting their content (e.g., advertisements) and the agents are assigned positions based, at least in part, on their bid amounts. Frequently, Web-based ads are presented in the form of a “banner ad,” which is a rectangular box on a Web page for inserting graphical elements. In the case when a Web page includes multiple ad slots, the system 100 may rank the slots in accordance with their bid value. For example, the system 100 can assign the ad slot that historically has the highest clickthrough or conversion rate to the highest bidder. In some implementations, the system 100, after a specified period of time, can determine costs imposed by a bidder on the next highest bidder and social values received from higher bidders. In this case, the system 100 can determine the imposed costs and social values based on the actual number of clicks that an advertiser receives in a specific position rather than using an expected number of clicks. In other words, the system 100 may determine charges in a position auction using actual conversions, i.e., independent of position normalizers.

In some implementations, the system 100 can charge, for each conversion, the cost imposed on the next highest bidder, i.e. and/or offset the charges with a social value added to bids of other bidders. For example, the system 100 can determine imposed cost based, at least in part, on the bid amount of the next highest bidder and/or the social value based, at least in part, on the difference between the bid amount and the next highest bidder's amount. In this example, the ad in that position associated with the conversion may not have received the response if the ads for higher bidders were not in their respective positions. In other words, removing one or more higher bidders can result in the ad moving to a different position, and as a result, can lead to losing the click or conversion. The system 100 assigns these higher bidders a payment, which, in some implementations, is subtracted from the imposed costs charged to the higher bidders.

At a high level, the system 100, in some implementations, includes a client 102 and a payment server 104 coupled through a network 106. In this implementation, the client 102 includes a Graphical User Interface (GUI) 110 for displaying Web pages provided by the payment server 104. The payment server 104 includes a memory 118 and a processor 120. In some implementations, the memory 118 locally stores tracking files 122 for identifying tracking information associated with ads, evaluation criteria 124 of identifying criteria for determining charges associated with ads, and billing files 126 for identifying the charges. The processor 120 includes a tracking engine 128 for tracking conversions associated with ads and an evaluation engine 130 for determining charges for presented ads using tracking files 122 and evaluation criteria 124. Turning to a high level description of the operation of the illustrated tracking system 100, the tracking engine 128 receives tracking information from the network 106 associated with ads and updates and/or generates one or more tracking files 122 based, at least in part, on the tracking information. In response to at least an event (e.g., request, period of time), the evaluation engine 130 identifies tracking files 122 and evaluation criteria 124 associated with a position auction and determines a charges associated with the ads using the tracking files 122 and the evaluation criteria 124. As mentioned above, the evaluation engine 130 may determine costs imposed on other bidders and social values associated with each bidder independent of position normalizers. Based, at least in part, on the determined charges, the evaluation engine 130 updates and/or generates one or more billing files 126.

In one specific implementation, the system 100 includes one or more clients 102 that includes or executes at least GUI 110 and comprises an electronic device (e.g., a computing device) operable to receive, transmit, process and store data associated with the system 100. While not illustrated, the system 100 may include any number of clients 102 communicably coupled to the payment server 104. Further, “client 102” and “user” may be used interchangeably as appropriate without departing from the scope of this disclosure. Moreover, for ease of illustration, the client 102 is described in terms of being used by one user, but many users may use one device (e.g., client 102) or that one user may use multiple devices without departing from the scope of this disclosure.

As used in this disclosure, the client 102 is any person, department, organization, small business, enterprise, or any other entity that may use or request others to use the system 100. The client 102 is intended to encompass a personal computer, touch screen terminal, workstation, network computer, kiosk, wireless data port, smart phone, personal data assistant (PDA), cellular device, one or more processors within these or other devices, or any other suitable electronic device used by a user viewing Web pages 112 from the payment server 104. For example, the client 102 may be a PDA operable to wirelessly connect with an external or unsecured network. In another example, the client 102 may comprise a laptop that includes an input device, such as a keypad, touch screen, mouse, or other device that can accept information, and an output device that conveys information from the payment server 104 (e.g., advertisements, search results), including digital data, visual information, or the GUI 110. Both the input device and output device may include fixed or removable storage media such as a magnetic computer disk, CD-ROM, or other suitable media to both receive input from and provide output to users of the client 102 through the display, namely the client portion of the GUI 110.

The GUI 110 comprises a graphical user interface operable to allow the user of the client 102 to interface with at least a portion of the system 100 for any suitable purpose, such as viewing Web pages 112 that may include advertisements, search results, hyperlinks to third-party Web sites, and other links. Generally, the GUI 110 provides the particular user with an efficient and user-friendly presentation of data provided by or communicated within the system 100. The GUI 110 may comprise a plurality of customizable frames or views having interactive fields, pull-down lists, and/or buttons operated by the user. For example, the GUI 110 is operable to display certain invoice information in a user-friendly form based on the user context and the displayed data. The term graphical user interface may be used in the singular or in the plural to describe one or more graphical user interfaces and each of the displays of a particular graphical user interface. The GUI 110 contemplates any graphical user interface, such as a generic web browser or touch screen, that processes information in the system 100 and presents the results to the user. The payment server 104 can accept data from the client 102 using, for example, the web browser (e.g., Microsoft Internet Explorer or Mozilla Firefox) and return the appropriate responses (e.g., HTML or XML) to the browser using the network 106.

The payment server 104 comprises an electronic device operable to receive, transmit, process and store data associated with the system 100. The system 100 can be implemented using devices or computers other than servers, as well as a server pool. Indeed, the payment server 104 may be any computer or processing device such as, for example, a blade server, general-purpose personal computer (PC), Macintosh, workstation, Unix-based computer, or any other suitable device. In other words, the system 100 may include computers other than general purpose computers as well as computers without conventional operating systems. In computing device implementations, the payment server 104 may be adapted to execute any operating system including Linux, UNIX, Windows Server, or any other suitable operating system.

As mentioned above, the payment server 104, in this implementation, includes the memory 118 and the processor 120. The memory 118 may be a local memory and include any memory or database module and may take the form of volatile or non-volatile memory including, without limitation, magnetic media, optical media, random access memory (RAM), read-only memory (ROM), removable media, or any other suitable local or remote memory component. In the illustrated implementation, the memory 118 includes the tracking files 122, the evaluation criteria 124, and the billing files 126. Local memory 118 may also include any other appropriate data such as VPN applications or services, firewall policies, a security or access log, print or other reporting files, HTML files or templates, data classes or object interfaces, child software applications or sub-systems, and others. Some portions of the memory 118 can be remote.

The tracking files 122 include one or more data structures or entries that identify or include information that identifies information associated with the presentation of ads. For example, one or more tracking files 122 may include or identify conversions associated with ads from a position auction. In some implementations, the tracking files 122 may identify or include information that identifies one or more of the following parameters: clicks, conversions, bid amounts, instances of impressions, bid rankings, ad positions, a time, date, user, client, a publisher, an advertiser, the creator of the ad, and/or a search engine The tracking file 122 may be associated with a specific position auction, multiple position auctions, a single advertiser, or multiple advertisers, or multiple tracking files 122 may be associated with a single position auction and/or a single advertiser. In some implementations, the association may be based on a parameter such as type of advertised product and/or service, demographic, region, or other parameters. For example, a first log file 418 may be associated with cleaning products and a second log file 418 may be associated with baseball paraphernalia. In some implementations, the tracking files 122 may be formatted, stored, or defined as various data structures in text files, XML documents, VSAM files, flat files, Btrieve files, CSV files, internal variables, or one or more libraries. In short, the tracking files 122 may comprise one table or file or a plurality of tables or files stored on one computer or across a plurality of computers in any appropriate format. Moreover, the tracking files 122 may be local or remote without departing from the scope of this disclosure and store any type of appropriate data.

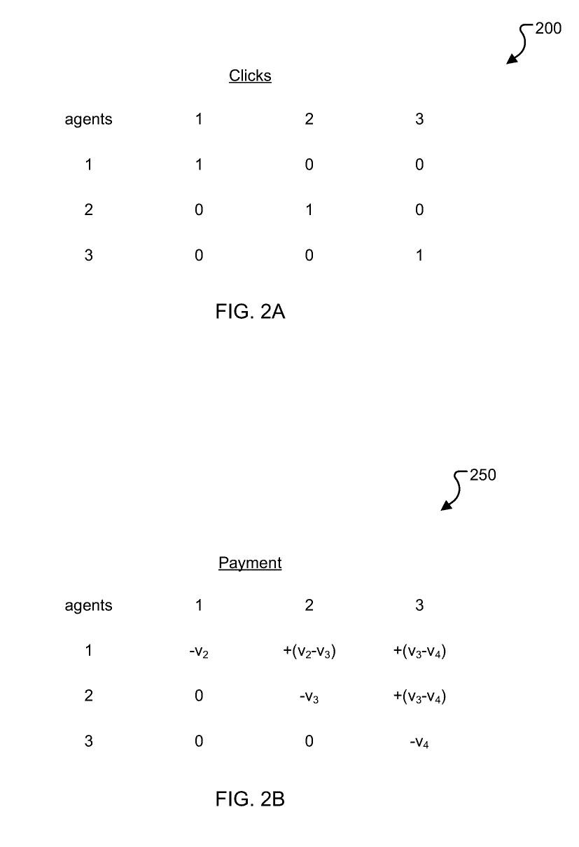

Evaluation criteria 124 include any parameters, variables, algorithms, instructions, rules, objects or other directives for determining charges for displaying ads in certain positions of Web pages. For example, evaluation criteria 124 may be used to map or otherwise associate certain ads with positions in Web pages and determine charges for the displaying the ads based, at least in part, on clicks, conversions, and/or other user actions. As mentioned above, such conversions may include one or more of the following: click throughs, revenue associated with adds, viewing time, and other interactions of a viewer associated with ads displayed in Web pages. In some implementations, conversions are the events of interest to an advertiser, e.g., purchasing a product, requesting further information, calling up a sales representative, etc.] In some implementations, the evaluation criteria 124 may include and/or identify criteria for determining one or more of the following: ranking agents in a position auction, assigning ads to positions in Web pages, determining conversions for the ads using the tracking files 122, determining cost imposed on other bidders, determining social values associated with bidders, determining charges to the agents, and/or generating or updating billing files 126 based on determinations. At a high level, the evaluation criteria 124 may include mathematical expressions for computing results (e.g., imposed costs, social values) of ads displayed in certain positions of Web pages based, at least in part, on associated conversions, bid amounts, and others. In terms of computing imposed costs, the evaluation criteria 124 may determine the cost imposed on other bidders (such as bidders whose ads may be displaced) when a click occurs for a displayed ad. In some implementations, the evaluation criteria 124 may include directives charging agent a an imposed cost vₐ₊₁, i.e., the value that the agent in position a+1 places on a click, for each received or expected click. If agent a was not present in the position auction, the agent a+1 may have received the click since agent a+1 would have been assigned the position on the Web page currently assigned to agent a. In regards to social values, the evaluation criteria 124 may include directives to subtract a social cost from a charge associated with an agent (e.g., vₐ−vₐ₊₁ from the bill of each agent a with bids greater than agent a). If agents bids higher than agent a's bid were not present in the position auction, the agent a may not have received the click since agent a would have been assigned a different position if one or more of the agents a were not present. Examples of adjusting charges using imposed costs and social values are discussed with respect to FIGS. 2A and 2B. In some specific implementations, there may be additional subtractions for each Though, these techniques are for illustration purposes only and the system 100 may include some, all, or none of the process describe above without departing from the scope of this disclosure.

In addition, the evaluation criteria 124 may include directives for incorporating effective click through rates (CTRs) associated with the agents. For example, the evaluation criteria 124 can include directives for estimating agent-specific effects and/or position effects in determining charges for presenting ads. In some implementations, the actual number of click received by an advertiser a in position p may be estimated as eₐxₚ where eₐ is an estimate of an agent-specific effect and xₚ is a position effect. Other formulations may also be used to relate the average position effect and the advertiser-specific effect to the final estimated clickthrough rates. In this example, the evaluation criteria 124 may include directives for ranking agents in accordance with eₐvₐ. In this case, the cost imposed on other bidders may be determined by the next highest bidders amount times the probability that the next highest bidder would have received the conversion. In other words, the evaluation criteria 124 may include directives for determining the cost imposed by agent a for receiving a conversion as v_{b+1}(e_{b+1}/e_{b}). In regards to social value, the evaluation criteria 124 may include directives for determining by v_{b}e_{b}−v_{b+1}(e_{b+1}/e_{b}). These techniques for determining imposed costs and social values using agent-specific effects are for illustration purposes only and system 100 may include some, all, or none of the process describe above without departing from the scope of this disclosure. In some implementations, the evaluation criteria 124 may include directives that dynamically adjust an estimate of position normalizers over time. For example, the evaluation criteria 124 may define a Bayesian model where xₚ is the prior expectation of the number of clicks per n impressions and zₚ is the actual number of clicks in n impressions. In the case that the total number of clicks at time T for position p is Z_{pT}, the evaluation criteria 124 may define a posterior estimate of the position normalizers as a_{T}Z_{pT}+(1−a_{T}) x_{pT}, where a_{T} represents a sample precision over prior precision plus sample precision. The evaluation criteria 124 can also include directives for estimating the position normalizer for position p by averaging 1/eₐ over the ads that appear in position p. The evaluation criteria 124 may also include a threshold such that if the difference between the estimates of the position normalizers exceed the threshold, this violation indicates an error.

The billing files 126 include one or more entries or data structures that identify or include information that identifies billing information associated with agents. For example, the billing file 126 may identify a summary of charges associated with a display ad. In addition, the billing file 126 includes other metrics associated with an advertisement such as a total number of click throughs, CTR, and others. In summary, the billing file 126 may include one or more of the following: a total charge, advertiser ID, imposed costs, social values, number of actions, type of action, and/or other information associated with the ads. The billing file 126 may be associated with a specific agent, multiple agents, a specific position auction, or multiple position auctions or multiple billing files 126 may be associated with a single agent and/or a single position auction. In some implementations, the billing files 126 may be formatted, stored, or defined as various data structures in text files, XML documents, VSAM files, flat files, Btrieve files, CSV files, internal variables, or one or more libraries. In short, billing files 126 may comprise one table or file or a plurality of tables or files stored on one computer or across a plurality of computers in any appropriate format. Moreover, the billing files 126 may be local or remote without departing from the scope of this disclosure and store any type of appropriate data.

Processor 120 executes instructions and manipulates data to perform operations of ad server 104. Although FIG. 1 illustrates a single processor 120 in server 104, multiple processors 120 may be used according to particular needs, and reference to processor 120 is meant to include multiple processors 120 where applicable. In the illustrated embodiment, processor 120 executes tracking engine 128 and evaluation engine 130 at any appropriate time such as, for example, in response to a request or input from a user of server 104 or any appropriate computer system coupled with network 106. Tracking engine 128 is any software, for example, operable to track actions associated with ads based on any suitable process. In general, tracking engine 128 may store information associated responses to an ad such as conversions. In connection with receiving information associated with an ad, tracking engine 128 may identify a tracking file 122 associated with the ad and store information in the tracking file 122. For example, tracking engine 128 may store one or more of the following in tracking file 122: conversions, bid amounts, instances of impressions, bid rankings, ad positions, a time, date, user, client, a publisher, an advertiser, and/or a search engine.

Evaluation engine 130 may determine charges associated with a position auction based on any suitable process. In some implementations, evaluation engine 130 may identify tracking information associated with ads in a position auction, determine imposed costs and/or social values associated with ads, and/or generate billing files 126 based on evaluations. In terms of identifying tracking information, the evaluation engine 130 may retrieve tracking information (e.g., number of clicks, bid amounts) from tracking files 122 associated with a position auction. In some implementations, the evaluation engine 130 identifies evaluation criteria 124 associated with the position auction and, using the evaluation criteria 124 and tracking information, determine charges (e.g., imposed costs, social values) for each agent assigned to a position in the position auction. For example, the evaluation engine 130 may identify the total number of clicks for each ad assigned a position in the ad auction, and using the number of clicks and bid amounts, determine imposed costs and social values for each agent. Based, at least in part, on these determinations, the evaluation engine 130 generates and/or updates billing files 126 associated with the agents.

Regardless of the particular implementation, “software” may include software, firmware, wired or programmed hardware, or any combination thereof as appropriate. Indeed, tracking engine 128 and evaluation engine 130 may be written or described in any appropriate computer language including C, C++, Java, J#, Visual Basic, assembler, Perl, any suitable version of 4GL, as well as others. It will be understood that while tracking engine 128 and evaluation engine 130 are illustrated in FIG. 1 as including individual modules, each of tracking engine 128 and evaluation engine 130 may include numerous other sub-modules or may instead be a single multi-tasked module that implements the various features and functionality through various objects, methods, or other processes. Further, while illustrated as internal to server 104, one or more processes associated with tracking engine 128 and/or evaluation engine 130 may be stored, referenced, or executed remotely. Moreover, tracking engine 128 and/or evaluation engine 130 may be a child or sub-module of another software module or enterprise application (not illustrated) without departing from the scope of this disclosure.

The payment server 104 may also include an interface 132 for communicating with other systems, such as the client 102, over the network 106 in a client-server or other distributed environment. In certain implementations, the payment server 104 receives data from internal or external senders through the interface 132 for storage in local memory 118 and/or processing by processor 120. Generally, the interface 132 comprises logic encoded in software and/or hardware in a suitable combination and operable to communicate with the network 106. More specifically, the interface 132 may comprise software supporting one or more communications protocols associated with communications over the network 106 or hardware operable to communicate physical signals.

The network 106 facilitates wireless or wireline communication between the payment server 104 and any other local or remote computer, such as the client 102. The network 106 may be all or a portion of an enterprise or secured network. While illustrated as single network, the network 106 may be a continuous network logically divided into various sub-nets or virtual networks without departing from the scope of this disclosure, so long as at least portion of the network 106 may facilitate communications between the payment server 104 and the client 102. In some implementations, the network 106 encompasses any internal or external network, networks, sub-network, or combination thereof operable to facilitate communications between various components in the system 100. The network 106 may communicate, for example, Internet Protocol (IP) packets, Frame Relay frames, Asynchronous Transfer Mode (ATM) cells, voice, video, data, and other suitable information between network addresses. The network 106 may include one or more local area networks (LANs), radio access networks (RANs), metropolitan area networks (MANs), wide area networks (WANs), all or a portion of the global computer network known as the Internet, and/or any other communication system or systems at one or more locations.

In one aspect of operation, the tracking engine 128 receives tracking information associated with a position auction and generates and/or updates tracking files 122 using the tracking information. In response to an event, the evaluation engine 130 can identify one or more tracking files 122 associated with the position auction and evaluation criteria 124. Using the tracking information, the evaluation engine 130 determines cost imposed on other agents and social values for each agent and aggregates the imposed costs and the social values to determine charges for each agent. The evaluation engine 130 can generate and/or update billing files 126 associated with the agents based, at least in part, on the determined charges.

FIGS. 2A and 2B illustrate example matrices 200 and 250, respectively, for determining payments during a billing certain time period independent of position normalizers. The illustrated matrices 200 and 250 are for example purposes only. Accordingly, the system 100 may include other features and functions for determining payments associated with a position auction without departing from the scope of the disclosure.

Referring to FIG. 2A, matrix 200 is a binary matrix (zₚₜ) associated with a position auction having 4 bidders bidding on 3 ad slots. In the illustrated example, the matrix 200 is associated with a billing period during which T clicks occurred and the parameter t=1, . . . , T indexes the clicks. The matrix 200 is generated by assigning zₚₜ=1 if click t has occurred in position p and zₚₜ=0 otherwise.

Referring to FIG. 2B, the matrix 250 illustrates the imposed cost and social values for each ad in the case each received a click. In regards to the first column, if agent 1 would not have been present, agent 2 would have occupied the first position. As a result, agent 2 would have received the click and yielding value v₂ to the agents other than agent 1. In other words, agent 1 imposed a cost on agent 2 equal to the value v₂. In regards to the second column, agent 2 receives a click, and as a result, agent 2 is charged v₃ since agent 3 would have received the click if agent 2 had not been present. If agent 1 were not present, agent 3 would have received the click agent 2 received. Accordingly, agent 1 contributes+(v₂−v₃) to the total value by virtue of his presence, so his charges are offset by the social value. Turning to the third column, agent 3 receives a click, so agent 3 is charged the cost imposed on agent 4, v₂. In addition, the agents 1 and 2 have increase their social value since they ensured that agent 3 received the click rather than agent 4. Accordingly, the charges for agents 1 and 2 are offset by the value+(v₃−v₄). Based, at least in part, on the matrix 250, the total payments for agents 1, 2, and 3 can be determined assuming that agent 1 received z₁ clicks, agent 2 received z₂ clicks, and agent 3 received z₃ clicks and is as follows:

payment 1: −v₂z₁+(v₂−v₃)z₂+(v₃−v₄)z₃=−v₂(z₁−z₂)−v₃(z₂−z₃)−v₄z₃

payment 2: −v₃z₂+(v₃−v₄)z₃=−v₃(z₂−z₃)−v₄z₃

payment 3: −v₄z₃=−v₄z₃

In this example, the payments for each agent is determined by the actual number of clicks that the advertiser in position p received during the billing period independent of position normalizers (e.g., expected number of clicks).

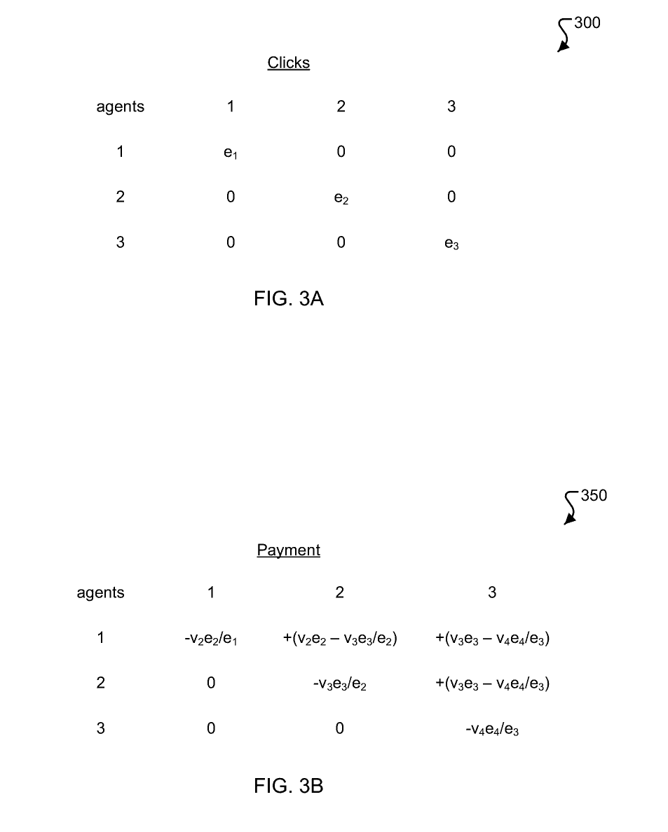

FIGS. 3A and 3B illustrate example matrices 300 and 350, respectively, for determining payments during a billing certain time period using an agent-specific effect. The illustrated matrices 300 and 350 are for example purposes only. Accordingly, the system 100 may include other features and functions for determining payments associated with a position −v₃z₂+(v₃−v₄)z₃=auction without departing from the scope of the disclosure.

The example method for determining payments in the case of 3 ad slots and 4 bidders illustrated in FIGS. 2A and 2B can, in some implementations, be extended to incorporate effective CTRs. For example, the actual number of clicks received by an advertiser a in position p can be assumed to be given by eₐxₚ, where eₐ is an estimated agent-specific effect and xₚ, is a position effect. In this case, the agents are ranked in accordance with the value of eₐvₐ. Referring to FIG. 3A, the matrices 300 is generated letting zₚₜ=eₐ, where eₐ is the effective CTR of the advertiser occurring in position p.

Referring to FIG. 3B, the matrices 350 illustrates the imposed cost and social values for each ad in the case each received a click. In regards to the first column, agent 1 receives a click, but in the absence agent 1 were absent, the probability that the click in position 1 would have occurred can be computed as e₂/e₁. For example, if agent 2 had one-third the effective CTR of agent 1, the click in position 1 would occur with a ⅓ probability. Based, at least in part, on the matrix 350, the payments for agents 1, 2, and 3 can be determined and are as follows:

payment 1: −v₂(e₂/e₁)z₁+(v₂e₂−v₃e₃/e₂)z₂+(v₃e₃−v₄e₄/e₃)z₃

payment 2: −v₃(e₃/e₂)z₂+(v₃e₃−v₄e₄/e₃)z₃

payment 3: −v₄(e₄/e₃)z₃

In this case, the position normalizers can be estimated by summing the clicks in each position and weighting by the predicted clickthrough rate of the ad that appeared in that position.

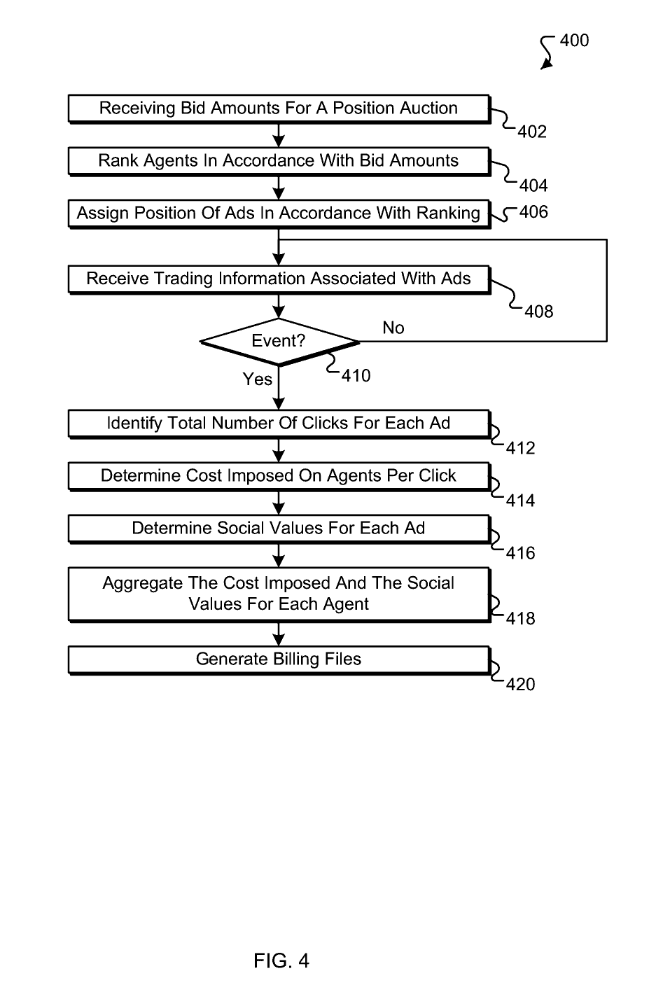

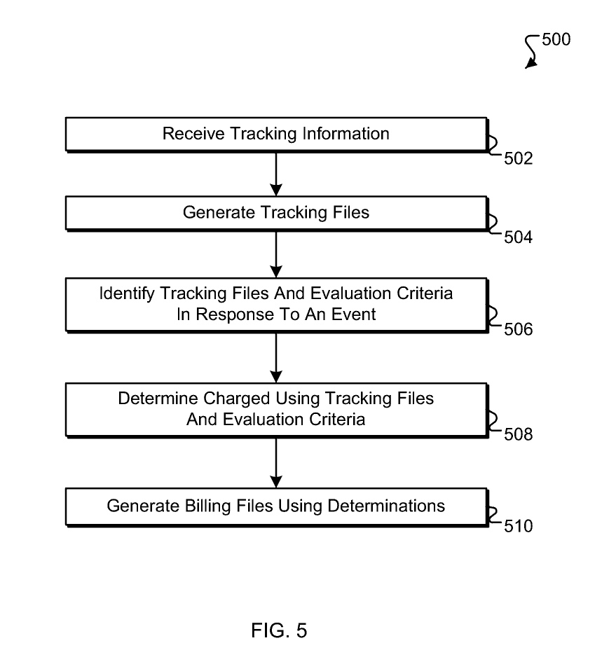

FIGS. 4 and 5 are flowcharts illustrating example methods 400 and 500, respectively, for determining charges in a position auction in accordance with some implementations of the present disclosure. Method 400 generally describes an example technique for determining imposed costs and social values for each agent independent of position normalizers. Method 500 generally describes an example for determining charges using tracking information. System 100 contemplates using any appropriate combination and arrangement of logical elements implementing some or all of the described functionality.

Referring to FIG. 4, the method 400 includes two processes: ranking bidders in a position bid at step 402 to 408; and determining charges for presenting ads in assigned positions at step 410 to 420. The method 400 begins at step 402 where bid amounts are received for a position auction. For example, four bids may be received for three ad slots in a Web page. At step 404, the agents associated with the bids are ranked according to their bid amounts. In the example, the four agents may be ranked from highest bidder to lowest bidder. Next, at step 406, the position of ads are assigned to the different ad slots in accordance with the ranking. Again in the example, the top three bidders are assigned to the 3 ad slots in the Web page. In this case, the highest bidder may be assigned to the ad slot that historically has the highest CTR. Tracking information associated with the presented ads is received at step 408. Turning to the example, conversions associated with the three ads are received.

Turning to the process for determining charges associated with the ads, if an event does not occur at decisional step 410, then execution returns to step 408. Otherwise, the total number of clicks for each ad is identified at step 412. Continuing with the example, the total number of clicks associated with each of the three ads is determined. At step 414, the cost impost on other agents per click is determined. In the example, the bid amount for the next highest bidder is charged to an agent for each click received. In connection with the cost, the social values are determined for each ad at step 416. Turning to the example, the social value can be determined by the difference in a bid amount and the next highest bid, and this difference is associated with each higher bidder. At step 418, the imposed costs and the social values for each agent are aggregated to determine the total charge for that agent. As for the example, the total imposed cost for each agent is offset by the total social values associated with the ad. Next, at step 420, a billing file is generated. In the example, an invoice can be generated for each agent detailing the imposed costs and the social values.

Referring to FIG. 5, the method 500 includes step 502 where tracking information is received. For example, the number of clicks per ad may be received. Using the tracking information, one or more tacking files are generated at step 504. In response to at least an event (e.g., expiration of a period of time), tracking files and evaluation criteria associated with a position auction are identified. Charges for presenting the ads are determining using the evaluation criteria to evaluate the tracking files at step 508. Next, at step 508, billing files associated with the agents are generated.

A number of embodiments of the invention have been described. Nevertheless, it will be understood that various modifications may be made without departing from the spirit and scope of the invention. Accordingly, other embodiments are within the scope of the following claims.

1. A method for charging advertisers, comprising:
identifying tracking information for a position auction associated with ads assigned to a plurality of positions in a first Web page and associated bid amounts from agents for each of the ads, the tracking information identifying interactions associated with the ads;
determining, by one or more computers, amounts to charge the agents using the tracking information and the bid amounts, the determining comprising, for each interaction identified by the tracking information:
determining an ad and an agent associated with the particular interaction,
determining a bid amount associated with a next highest bid from the agents,
determining a charge for the agent associated with the particular interaction based on the bid amount associated with the next highest bid from the agents,
identifying higher-bidding agents among the agents that bid more than the agent associated with the particular interaction, and
for each of the higher-bidding agents, determining a social value based on a value to each higher-bidding if the agent associated with the particular interaction were not present in the position auction;
generating invoices for the agents, wherein for each of the agents, the charges are offset by the social values;
providing the invoices for display to the agents in one or more graphical user interfaces.

2. The method of claim 1, further comprising:
receiving the bid amounts from the agents in response to a position auction; and
ranking the ads associated with the agents in accordance with the bid amounts.

3-8. (canceled)

9. The method of claim 1, further comprising:
identifying an agent-based effect associated with each agent; and
wherein the charges are based, at least in part, on the agent-based effect.

10. A computer-readable medium having instructions stored thereon, which, when executed by a processor, cause the processor to perform operations comprising
identifying tracking information for a position auction associated with ads assigned to a plurality of positions in a first Web page and associated bid amounts from agents for each of the ads, the tracking information identifying interactions associated with the ads;
determining amounts to charge the agents using the tracking information and the bid amounts, the determining comprising, for each interaction identified by the tracking information:
determining an ad and an agent associated with the particular interaction,
determining a bid amount associated with a next highest bid from the agents,
determining a charge for the agent associated with the particular interaction based on the bid amount associated with the next highest bid from the agents,
identifying higher-bidding agents among the agents that bid more than the agent associated with the particular interaction, and
for each of the higher-bidding agents, determining a social value based on a value to each higher-bidding if the agent associated with the particular interaction were not present in the position auction;
generating invoices for the agents, wherein for each of the agents, the charges are offset by the social values;
providing the invoices for display to the agents in one or more graphical user interfaces.

11. The computer-readable medium of claim 10, the operations further comprising:
receiving the bid amounts from the agents in response to a position auction; and
ranking the ads associated with the agents in accordance with the bid amounts

12-17. (canceled)

18. The computer-readable medium of claim 10, the operations further comprising identifying an agent-based effect associated with each agent, wherein the charges are based, at least in part, on the agent-based effect.

19. A server for charging advertisers comprising one or more processors operable to:
identify tracking information for a position auction associated with ads assigned to a plurality of positions in a first Web page and associated bid amounts from agents for each of the ads, the tracking information identifying interactions associated with the ads;
determine amounts to charge the agents using the tracking information and the bid amounts, the determining comprising, for each interaction identified by the tracking information:
determining an ad and an agent associated with the particular interaction,
determining a bid amount associated with a next highest bid from the agents,
determining a charge for the agent associated with the particular interaction based on the bid amount associated with the next highest bid from the agents,
identifying higher-bidding agents among the agents that bid more than the agent associated with the particular interaction, and
for each of the higher-bidding agents, determining a social value based on a value to each higher-bidding if the agent associated with the particular interaction were not present in the position auction;
generate invoices for the agents, wherein for each of the agents, the charges are offset by the social values;
provide the invoices for display to the agents in one or more graphical user interfaces.

20. The server of claim 19, further operable to:
receive the bid amounts from the agents in response to a position auction; and
rank the ads associated with the agents in accordance with the bid amounts

21-31. (canceled)

32. The method of claim 1, wherein determining a social value comprises determining a difference between a bid amount associated with the particular higher-bidding agent and a bid amount associated with the particular interaction.

33. The computer-readable medium of claim 10, wherein determining a social value comprises determining a difference between a bid amount associated with the particular higher-bidding agent and a bid amount associated with the particular interaction.

34. The server of claim 19, wherein determining a social value comprises determining a difference between a bid amount associated with the particular higher-bidding agent and a bid amount associated with the particular interaction.
